(19) United States
(12) Patent Application Publication
(10) Pub. No.: US 2026/0027335 A1
(43) Pub. Date: Jan. 29, 2026

(54) APPARATUS FOR VAGINAL CLEANSING, VAGINAL DRUG DELIVERY, AND TOXIC SHOCK PREVENTION

(71) Applicant: Angela Jean Yonce, Petal, MS (US)

(72) Inventor: Angela Jean Yonce, Petal, MS (US)

(21) Appl. No.: 19/295,553

(22) Filed: Aug. 9, 2025

(51) Int. Cl. A61M 31/00; A61F 13/20; A61F 13/26; A61F 13/84; A61K 9/00; A61K 31/155; A61K 31/19; A61K 31/4164; A61K 31/522

(52) U.S. Cl. CPC A61M 31/002; A61F 13/2074; A61F 13/266; A61F 13/8405; A61K 9/0036; A61K 31/155; A61K 31/19; A61K 31/4164; A61K 31/522; A61F 2013/8408; A61F 2013/8497; A61M 2205/3324; A61M 2205/6081; A61M 2210/1475

(57) ABSTRACT

An intravaginal apparatus comprising a bullet-shaped body with outer, middle mesh, and inner layers is provided. The device is configured to contain insertable materials such as tampons, absorbents, drugs, or therapeutic compounds. One or more openings, optionally fitted with mesh guards, enable fluid exchange and adsorption of pathogens and odor-causing compounds. In some embodiments, the insertable material includes activated carbon engineered to adsorb volatile amines and indoxyl sulfate and may release therapeutic agents in response to microbial presence. The apparatus may preserve vaginal pH and microbiota, support directional fluid flow, and be suitable for single-use or extended wear. Applications include treatment and prevention of vaginal infections such as bacterial vaginosis and toxic shock syndrome.

### FIELD OF THE INVENTION

The present invention relates to intravaginal medical devices designed for therapeutic, prophylactic, and hygienic applications. More specifically, it pertains to an apparatus configured to contain and deliver insertable materials capable of adsorbing vaginal odor compounds, trapping and neutralizing pathogens, and optionally releasing therapeutic agents, while preserving mucosal integrity and microbiota balance.

### BACKGROUND OF THE INVENTION

Vaginal infections such as bacterial vaginosis (BV), herpes simplex virus (HSV), and toxic shock syndrome (TSS) are prevalent conditions affecting millions globally. Conventional treatments often rely on systemic antibiotics or antivirals, which may disrupt the vaginal microbiome or contribute to antimicrobial resistance. There is a growing need for localized, biocompatible, and multifunctional devices that address odor, infection, and microbial imbalance without compromising mucosal health.

Activated carbon is widely recognized for its adsorptive properties. Its use in intravaginal contexts has shown promise in reducing odor and preserving beneficial lactobacilli. The integration of porous carbonaceous materials, whether unbound, freely associating, or encapsulating therapeutic agents offer a novel pathway for localized treatment and prevention of vaginal disorders.

### SUMMARY OF THE INVENTION

The invention provides an intravaginal apparatus comprising a bullet-shaped body formed of a flexible, stretchable, or nonflexible material. The body includes an outer layer, a middle mesh layer (optional), and an inner layer. The apparatus is configured to contain one or more insertable materials selected from a group consisting of tampons, medicated tampons, absorbent materials, nonabsorbent materials, adsorbents, drugs, herbs, chemicals, compounds, smaller devices, and smaller apparatuses.

The device includes one or more openings, optionally fitted with mesh guards, to facilitate fluid exchange and enable adsorption of odor-causing compounds and pathogens. In some embodiments, the insertable material comprises a porous carbonaceous substance, which may be unbound, freely associating, or embedded within a matrix. These materials may be configured to selectively adsorb volatile amines, sulfur compounds, and microbial toxins. Therapeutic agents may be encapsulated within the carbon matrix and released in response to environmental triggers such as microbial contact, pH variation, or fluid saturation.

The apparatus may be configured for single-use or extended wear, and may include optional features such as colorimetric indicators, modular inserts, and batch-coded components for lifecycle traceability.

### BRIEF DESCRIPTION OF THE DRAWINGS

FIG. 1 illustrates a bullet-shaped body of the apparatus with mesh-screened openings.

FIG. 2 illustrates a bullet-shaped body of the apparatus with non-mesh openings.

FIG. 3 illustrates a cross-sectional side view of the apparatus (shown in FIG. 1), showing the outer layer, middle mesh layer, and inner layer

### DETAILED DESCRIPTION OF THE DRAWINGS

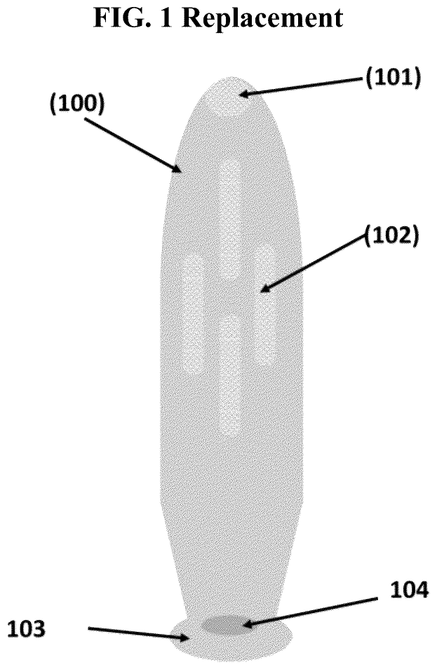

FIG. 1 illustrates a bullet-shaped apparatus featuring; (100) outer body designed to fit comfortably in the vagina; (101) top-round mesh-screened opening designed to facilitate fluid and drug exchange and adsorption function. (102) side-oval mesh-screened opening designed to facilitate fluid and drug exchange and adsorption function; (103) outer lip (rim) to hold the apparatus in place; (104) opening to inner chamber of the apparatus designed for the insertion of device, material, compound, drug, etc.,

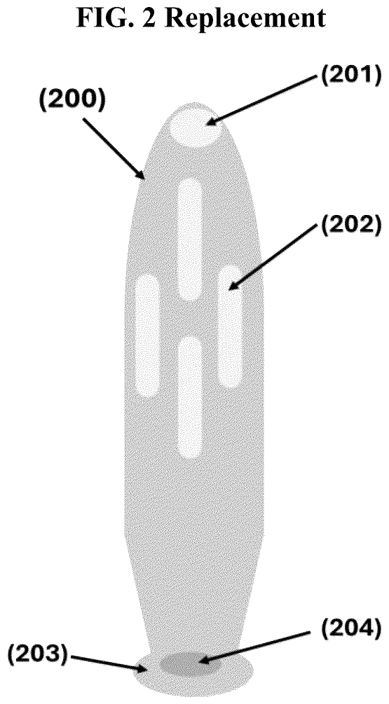

FIG. 2 illustrates a bullet-shaped outer body of the apparatus featuring; (200) outer body designed to fit comfortably in the vagina; (201) top-round opening (without mesh) designed to facilitate fluid and drug exchange and adsorption function. (202) side-oval opening (without mesh) designed to facilitate fluid and drug exchange and adsorption function; (203) outer lip (rim) to hold the apparatus in place; (204) opening to inner chamber of the apparatus designed for the insertion of device, material, compound, drug, etc.,

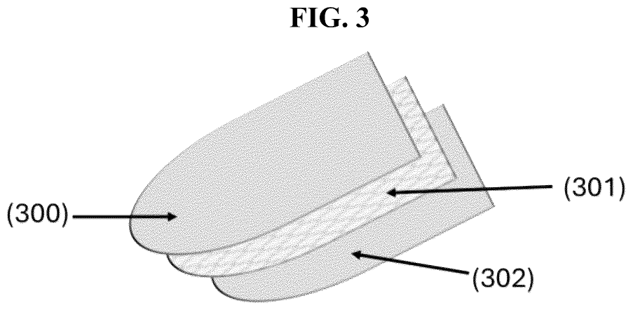

FIG. 3 illustrates a cross-sectional side view of the apparatus (shown in FIG. 1) showing the layered structure, including the outer layer (300), middle mesh layer (301), and inner layer (302).

### DETAILED DESCRIPTION OF THE INVENTION

The intravaginal apparatus comprises a multi-layered structure designed to house and deliver insertable materials with therapeutic or prophylactic function. The outer layer may be constructed from a hydrophobic polymer to minimize external fluid ingress, while the inner layer may be hydrophilic to facilitate directional fluid flow. The middle mesh layer serves as a containment scaffold and may be composed of biocompatible polymers such as polyethylene, polyurethane, or silicone. In certain embodiments, the middle mesh layer is present only in configurations featuring mesh-screened openings. This layer functions as a containment scaffold and fluid exchange screen, and may be omitted in embodiments with non-mesh openings. The structural variation allows for tailored use depending on the therapeutic or hygienic material being housed within the apparatus.

Insertable materials may include unbound or freely associating porous carbonaceous substances, such as powdered, granular, or spherical activated and/or non-activated carbon. These materials are selected for their high surface area and pore volume, enabling selective adsorption of odor compounds and microbial toxins. In certain embodiments, the carbonaceous material may be functionalized to encapsulate therapeutic agents, forming drug-encapsulated carbon matrices. These agents may include antivirals, antibiotics, or anti-inflammatory compounds, and may be released in a sustained or stimulus-responsive manner.

In certain embodiments, the activated carbon may be spherical in shape and exhibit a surface area exceeding 1000 m²/g, with residual metal content below 0.01%. These properties may enhance adsorption efficiency, reduce mucosal irritation, and improve therapeutic payload stability. Other forms and purities of carbon may also be used depending on the desired therapeutic profile.

The apparatus may also contain absorbent or nonabsorbent substrates, medicated tampons, herbs, chemicals, or smaller devices. The device may be configured for short-term use (e.g., 8-12 hours) or extended wear (e.g., 3-7 days), depending on the therapeutic load and material properties.

In certain embodiments, the insertable material may be placed directly within the apparatus or enclosed in a cartridge, depending on the physical characteristics, dosage requirements, or therapeutic profile of the material. Direct placement may be suitable for textiles, tampons, or solid compounds, while cartridges may be used for powders, liquids, or sensitive agents requiring containment. This configuration allows for adaptable use across hygienic and therapeutic applications.

Functionally, the device is designed to house smaller devices, material, herbs, drugs, and adsorbents; for the adsorption of vaginal odor compounds such as trimethylamine and putrescine, trap pathogens, and optionally deliver therapeutic agents locally. The carbon/charcoal materials may also adsorb indoxyl sulfate and similar toxins, mimicking systemic detoxification mechanisms. The device supports the preservation of hydrogen peroxide (producing lactobacilli) and maintains vaginal pH within a healthy range, thereby reducing susceptibility to dysbiosis and infection.

In certain embodiments, the apparatus is configured to function as a protective housing or containment shell for insertable materials such as tampons, medicated tampons, cotton balls, or therapeutic textiles. This configuration provides added protection to the vaginal walls and enables controlled, localized administration of drugs, herbs, or other compounds. The apparatus may be inserted into the vaginal canal with the insertable material enclosed, allowing for sustained contact, directional fluid exchange, and targeted therapeutic delivery.

Applications include treatment and prevention of BV, HSV, TSS, and general menstrual hygiene; for the adsorption, entrapment, and extraction of odor-causing compounds, bacteria, yeast, parasites, and viruses.

Optional features may include colorimetric indicators for pH or pathogen presence, modular inserts for personalized therapy, and batch-coded components for regulatory traceability.

I claim:

1. An intravaginal apparatus comprising:
a bullet-shaped body formed of a flexible, stretchable, or nonflexible biocompatible material;
an outer layer configured to interface with the vaginal wall and repel external moisture;
a middle mesh layer, present only in embodiments comprising mesh-screened openings, configured to interface with the vaginal wall and support directional fluid flow and particulate retention;
an inner layer configured to contain and support insertable materials without direct mucosal contact;
one or more openings configured to enable bidirectional fluid exchange and containment of odor-causing compounds or pathogens, the openings optionally fitted with mesh guards and selected based on the physical properties of the insertable material;
wherein the apparatus is configured to house and position an insertable material selected from the group consisting of: a tampon, a medicated tampon, a cotton ball, a therapeutic textile, a hygienic textile, an absorbent material, a nonabsorbent material, an adsorbent material, a drug, a herb, a chemical, a compound, a smaller device, and a smaller apparatus;
and wherein the insertable material is housed within the apparatus either directly or within a cartridge, depending on the physical or therapeutic properties of the material;
and wherein the apparatus provides physical protection to the vaginal walls and enables controlled administration of the insertable material.

2. The apparatus of claim 1, wherein the cartridge is removable and configured for tool-free replacement.

3. The apparatus of claim 1, wherein the device supports maintenance of vaginal pH balance.

4. The apparatus of claim 1, wherein the outer layer is hydrophobic and the inner layer is hydrophilic, configured to direct fluid inward while minimizing backflow.

5. The apparatus of claim 1, wherein the device is configured for single-use or multi-day wear with sustained containment and fluid exchange.

6. The apparatus of claim 1, wherein the openings are sized to permit fluid exchange while retaining particulate adsorbents.

7. The apparatus of claim 1, wherein the device selectively preserves vaginal lactobacilli while facilitating the containment of anaerobic bacteria and pathogenic yeasts.

8. The apparatus of claim 1, wherein the device includes a visual indicator responsive to saturation levels or pH changes, comprising a colorimetric strip or embedded sensor.

9. The apparatus of claim 1, wherein the device is configured to house therapeutic or hygienic textiles for sustained local administration.

10. The apparatus of claim 1, wherein the device includes modular inserts for personalized therapy or hygiene applications.

11. An intravaginal apparatus comprising:
an insertable material housed within the apparatus, either directly or within a cartridge, the insertable material comprising an activated carbon-based adsorbent configured to adsorb vaginal odor compounds and trap pathogens;
wherein the apparatus is configured to contain and position the insertable material for sustained contact with vaginal mucosa while maintaining separation via the inner layer.

12. The apparatus of claim 11, wherein the activated carbon is spherical and optimized for adsorption of indoxyl sulfate, trimethylamine, and volatile amines.

13. The apparatus of claim 11, wherein the activated carbon is impregnated with a therapeutic agent selected from the group consisting of: acyclovir, metronidazole, lactic acid, and chlorhexidine.

14. The apparatus of claim 11, wherein the therapeutic agent is encapsulated within the activated carbon matrix and released upon contact with microbial triggers.

15. The apparatus of claim 11, wherein the therapeutic agent is released via stimulus-responsive mechanisms involving enzymatic cleavage, pH shift, or temperature change.

16. The apparatus of claim 11, wherein the activated carbon adsorbs volatile amines and sulfur-containing compounds associated with vaginal dysbiosis while maintaining mucosal hydration.

17. The apparatus of claim 11, wherein the activated carbon selectively adsorbs odor-causing compounds without disrupting beneficial microbial populations.

18. The apparatus of claim 11, wherein the activated carbon is derived from a high-purity spherical carbon adsorbent with minimal residual metal content and high surface area.

19. The apparatus of claim 2, wherein the therapeutic agent is released in a sustained manner over 8 to 72 hours depending on fluid saturation rate.
The apparatus of claim 11, wherein the therapeutic agent is released in a sustained manner over 8 to 72 hours depending on fluid saturation rate and microbial load.

20. A method of administering a vaginally delivered therapeutic or hygienic material comprising:
inserting the apparatus of claim 1 into the vaginal canal;
wherein the apparatus contains an insertable material selected from the group consisting of: a tampon, a medicated tampon, a cotton ball, a therapeutic textile, a hygienic textile, a drug, a herb, or an activated carbon compound;
and wherein the insertable material is housed within the apparatus either directly or within a cartridge, depending on the material's physical or therapeutic properties;
and wherein the apparatus provides physical protection to the vaginal walls and facilitates sustained contact and controlled administration of the insertable material.
